(19) United States
(12) Patent Application Publication
(10) Pub. No.: US 2026/0127898 A1
(43) Pub. Date: May 7, 2026

(54) APPARATUS FOR ACQUIRING STOCK INFORMATION OF MEDICINES AND METHOD THEREOF

(71) Applicant: Sung An Byun, Gyeonggi-do (KR)

(72) Inventor: Sung An Byun, Gyeonggi-do (KR)

(21) Appl. No.: 19/377,321

(22) Filed: Nov. 3, 2025

(30) Foreign Application Priority Data
Nov. 6, 2024 (KR) 10-2024-0156007

(51) Int. Cl. G06V 20/64; G06V 10/24; G06V 10/75; G06V 20/62; G16H 40/20

(52) U.S. Cl. CPC G06V 20/64; G06V 10/242; G06V 10/751; G06V 20/62; G16H 40/20

(57) ABSTRACT

According to the present disclosure, disclosed is a method of acquiring information by an electronic apparatus, which includes acquiring a target image, identifying one or more medicines included in the target image, and acquiring, based on a result obtained by identifying the one or more medicines, stock information for each medicine type included in the target image.

### CROSS-REFERENCE TO RELATED APPLICATION(S)

This application claims the benefit of Korean Patent Application No. 10-2024-0156007, filed on Nov. 6, 2024, in the Korean Intellectual Property Office, the disclosure of which is incorporated herein by reference.

### BACKGROUND

### 1. Field of the Invention

The present disclosure relates to an apparatus for acquiring stock information of medicines and a method thereof. More specifically, the present disclosure relates to an electronic apparatus, which identifies one or more medicines included in an acquired target image and acquires, based on a result of the identification, stock information for each medicine type included in the target image, and a method thereof.

### 2. Description of the Related Art

In the case of medicines, it is particularly required to manage stock thoroughly due to their characteristics, such as a point in which customers may experience great inconvenience when a medicine is not provided in time, a point in which health damage may be caused when a wrong medicine is provided, and a point in which some medicines may be used for undesirable behavior like abuse when the medicines are wrongly released.

Meanwhile, as types and quantities of medicines that are required to be stored and managed in a pharmacy continue to grow due to development of medical treatment technology and pharmaceutical technology, considerable costs and effort are consumed to manage stock of medicines.

In relation to this, prior documents such as KR101674195B1 and KR102245345B1 may be referred to.

### SUMMARY

A task to be solved by an example embodiment is, for solving the above-mentioned issue, to provide an electronic apparatus, which acquires a target image, identifies one or more medicines included in the target image, and acquires, based on a result obtained by identifying the one or more medicines, stock information for each medicine type included in the target image, and a method thereof.

A technical task to be achieved by an example embodiment of the present disclosure is not limited to the technical task described above, and other technical tasks may be derived from example embodiments described below.

A method of acquiring information according to an example embodiment includes acquiring a target image, identifying one or more medicines included in the target image, and acquiring, based on a result obtained by identifying the one or more medicines, stock information for each medicine type included in the target image.

According to an example embodiment, the identifying of the one or more medicines may include identifying one or more feature components included in the target image and acquiring type information of a medicine related to a first feature component included in the one or more feature components.

According to an example embodiment, the acquiring of the type information of the medicine related to the first feature component may include comparing the first feature component with one or more comparative images.

According to an example embodiment, the comparing of the first feature component with the one or more comparative images may include inputting the first feature component and the one or more comparative images to a pre-trained machine learning model and acquiring an output of the machine learning model. The machine learning model may be pre-trained based on a plurality of medicine images.

According to an example embodiment, the comparing of the first feature component with the one or more comparative images may include determining one or more attributes related to the first feature component and selecting, based on the one or more attributes, the one or more comparative images from a plurality of images included in a medicine image group.

According to an example embodiment, the determining of the one or more attributes may include identifying logo information included in the first feature component.

According to an example embodiment, the acquiring of the type information of the medicine related to the first feature component may include acquiring gradient information related to the first feature component and rotating the first feature component based on the gradient information.

According to an example embodiment, the acquiring of the type information of the medicine related to the first feature component may include acquiring distortion information related to the first feature component and correcting the first feature component based on the distortion information. The correcting of the first feature component may include performing warping for the first feature component.

According to an example embodiment, the method of acquiring information may further include acquiring photographing environment information of the target image. The identifying of the one or more medicines may include transforming the target image based on the photographing environment information.

According to an example embodiment, the photographing environment information may include one or more pieces of product information of a camera for photographing the target image, setting information of the camera when the target image is photographed, distance information between the camera and a target to be photographed when the target image is photographed, and angle information between the camera and the target to be photographed when the target image is photographed.

According to an example embodiment, the acquiring of the stock information may include determining types of the plurality of medicines existing in a space corresponding to the target image and acquiring position information of the plurality of medicines.

According to an example embodiment, the method of acquiring information may further include acquiring, based on the stock information, three-dimensional (3D) modeling information corresponding to the target image.

According to an example embodiment, the acquiring of the 3D modeling information may include generating one or more geometric models corresponding to at least a portion of the plurality of medicines existing in a space corresponding to the target image and performing a texture mapping process for the one or more geometric models.

According to an example embodiment, the method of acquiring information may further include acquiring storage environment information of the one or more medicines. The electronic apparatus may acquire the stock information based on the storage environment information.

According to an example embodiment, the storage environment information may include one or more pieces of attribute information related to a storage part for storing the one or more medicines. The one or more pieces of attribute information may include one or more pieces of information for product identification of the storage part, shape information of the storage part, size information of the storage part, and volume information of the storage part.

According to an example embodiment, the method of acquiring information may further include acquiring, based on the storage environment information and the stock information, suggestion information related to a storage method of the one or more medicines.

According to an example embodiment, the method of acquiring information may further include comparing the stock information with pre-existing computerized stock information.

According to an example embodiment, when there is a discrepancy between the stock information and the pre-existing computerized stock information, the method of acquiring information may further include notifying the discrepancy.

An electronic apparatus acquiring information according to an example embodiment may include a memory that stores an instruction and a processor. The processor, which is connected to the memory, may be configured to acquire a target image, identify one or more medicines included in the target image, and acquire, based on a result obtained by identifying the one or more medicines, stock information for each medicine type included in the target image.

Specific details of other embodiments are included in the detailed description and the drawings.

### BRIEF DESCRIPTION OF THE DRAWINGS

These and/or other aspects, features, and advantages of the invention will become apparent and more readily appreciated from the following description of example embodiments, taken in conjunction with the accompanying drawings of which:

FIG. 1 is a block diagram illustrating a system that manages stock information according to an example embodiment;

FIG. 2 is a diagram for describing a method of acquiring, by an electronic apparatus, stock information of medicines according to an example embodiment;

FIG. 3 is a diagram for describing an outer package of a medicine according to an example embodiment;

FIG. 4 is a diagram for describing a main display surface of a medicine according to an example embodiment;

FIG. 5 is a diagram for describing an information display surface of a medicine according to an example embodiment;

FIG. 6 is a diagram for describing photographing environment information of a target image according to an example embodiment;

FIG. 7 is a diagram for describing a method of storing medicines according to an example embodiment;

FIG. 8 is a diagram for describing a method of storing medicines according to an example embodiment;

FIG. 9 is a diagram for describing 3D modeling information according to an example embodiment; and

FIG. 10 is an exemplary view of a configuration of an electronic apparatus according to an example embodiment.

### DETAILED DESCRIPTION

Terms used in the example embodiments are selected, as much as possible, from general terms that are widely used at present while taking into consideration the functions obtained in accordance with the present disclosure, but these terms may be replaced by other terms based on intentions of those skilled in the art, customs, emergence of new technologies, or the like. Also, in a particular case, terms that are arbitrarily selected by the applicant of the present disclosure may be used. In this case, the meanings of these terms may be described in corresponding description parts of the disclosure. Accordingly, it should be noted that the terms used herein should be construed based on practical meanings thereof and the whole content of this specification, rather than being simply construed based on names of the terms.

In the entire specification, when a component is referred to as “including” another component, the component should not be understood as excluding other components so long as there is no special conflicting description, and the component may include at least one other component. In addition, the terms “unit” and “module”, for example, may refer to a component that exerts at least one function or operation, and may be realized in hardware or software, or may be realized by combination of hardware and software.

The expression “at least one of A, B, and C” may indicate the following meaning including: A alone; B alone; C alone; both A and B together; both A and C together; both B and C together; or all three of A, B, and C together.

In the present disclosure, a “terminal” may be implemented as a computer or a portable terminal capable of accessing a server or another terminal through a network. Here, the computer may include, for example, a laptop computer, a desktop computer, and a notebook equipped with a web browser. The portable terminal may be a wireless communication device ensuring a portability and a mobility, and include any type of handheld wireless communication device, for example, a tablet PC, a smartphone, a communication-based terminal such as international mobile telecommunication (IMT), code division multiple access (CDMA), W-code division multiple access (W-CDMA), and long term evolution (LTE).

In the following description, example embodiments of the present disclosure will be described in detail with reference to the drawings so that those skilled in the art can easily carry out the present disclosure. However, the present disclosure may be embodied in many different forms and is not limited to the embodiments described herein.

The following description of example embodiments disclosed in the present disclosure is provided with reference to the accompanying drawings.

In describing the example embodiments, descriptions of technical contents that are well known in the art to which the present disclosure belongs and are not directly related to the present specification will be omitted. This is to more clearly communicate without obscuring the subject matter of the present specification by omitting unnecessary description.

For the same reason, in the accompanying drawings, some components are exaggerated, omitted, or schematically illustrated. In addition, the size of each component does not fully reflect the actual size. The same or corresponding components in each drawing are given the same reference numerals.

Advantages and features of the present disclosure and methods of achieving them will be apparent from the following example embodiments that will be described in more detail with reference to the accompanying drawings. It should be noted, however, that the present disclosure is not limited to the following example embodiments, and may be implemented in various forms. Accordingly, the example embodiments are provided only to disclose the present disclosure and let those skilled in the art know the category of the present disclosure, and the present disclosure is only defined by the category of claims. The same reference numerals or the same reference designators denote the same elements throughout the specification.

At this point, it will be understood that each block of the flowchart illustrations and combinations of flowchart illustrations may be performed by computer program instructions. Since these computer program instructions may be mounted on a processor of a general purpose computer, special purpose computer, or other programmable data processing equipment, those instructions executed through the computer or the processor of other programmable data processing equipment may create a means to perform the functions described in flowchart block(s). These computer program instructions may be stored in a computer usable or computer readable memory that can be directed to a computer or other programmable data processing equipment to implement functionality in a particular manner, and thus computer program instructions stored in the computer usable or computer readable memory can produce an article of manufacture containing instruction means for performing the functions described in the flowchart block(s). Computer program instructions may also be mounted on a computer or other programmable data processing equipment, such that a series of operating steps may be performed on the computer or other programmable data processing equipment to create a computer-implemented process to create a computer or other programmable data. Instructions for performing the computer or other programmable data processing equipment may also provide steps for performing the functions described in the flowchart block(s).

In addition, each block may represent a portion of a module, segment, or code that includes one or more executable instructions for executing a specified logical function(s). It should also be noted that in some alternative implementations, the functions noted in the blocks may occur out of order. For example, the two blocks shown in succession may in fact be executed substantially concurrently, or the blocks may sometimes be executed in the reverse order, depending on the corresponding function.

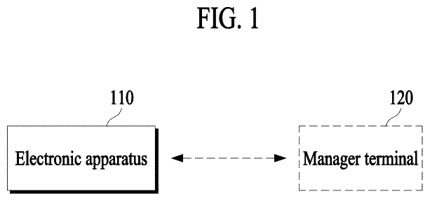

FIG. 1 is a block diagram illustrating a system that manages stock information according to an example embodiment.

According to various example embodiments, the system that manages stock information includes an electronic apparatus 110. The system that manages stock information according to an example embodiment may further include a manager terminal 120. The system that manages stock information according to an example embodiment may further include the electronic apparatus 110, the manager terminal 120, and a network that supports information transmission and reception between at least a portion of external devices.

The electronic apparatus 110 and the manager terminal 120 of the system that manages stock information may include a memory and a processor and may further include a transceiver according to an example embodiment. In addition, each of the electronic apparatus 110 and the manager terminal 120 may refer to a unit processing at least one function or operation, which may be implemented as hardware, software, or a combination of hardware and software.

Meanwhile, in the overall example embodiments, the electronic apparatus 110 and the manager terminal 120 are referred to as a separate apparatus and server, but this may be a logically divided structure and may be implemented by functions separated from a single apparatus or server. For example, according to an example embodiment, operations described as being performed by the manager terminal 120 may be actually performed by the electronic apparatus 110 and, in this case, an operation of transmitting and receiving information between the electronic apparatus 110 and the manager terminal 120 may be understood actually as an operation of exchanging data inside the electronic apparatus 110.

According to an example embodiment, the electronic apparatus 110 and the manager terminal 120 may include multiple computer systems or computer software implemented by a network server. For example, at least a portion of the electronic device 110 and the manager terminal 120 may refer to a computer system and computer software that are connected to sub-devices capable of communicating with other network servers through a computer network such as an intranet or the Internet to receive a task execution request, perform the corresponding task, and provide a result of the execution. In addition, at least a portion of the electronic apparatus 110 and the manager terminal 120 may be understood in a broad sense as a concept including a series of application programs capable of operating on a network server and various databases constructed inside. For example, at least a portion of the electronic apparatus 110 and the manager terminal 120 may be implemented by using network server programs provided in various ways according to an operating system such as DOS, Windows, Linux, UNIX, or macOS.

Meanwhile, for convenience of description, each operation subject is referred to as the electronic apparatus 110 and the manager terminal 120, which should be, however, understood as comprehensive forms of devices that may correspond to various types of devices such as a computer device and a mobile communication terminal, may include various types of devices, or may be included in various types of devices.

The electronic apparatus 110 is an apparatus that acquires and processes various pieces of information. In addition, the electronic apparatus 110 may provide various pieces of information. The electronic apparatus 110 may perform various tasks for checking (for example, acquiring stock information) and managing stock of medicines.

In relation to an operation of the electronic apparatus 110, more specifically, the electronic apparatus 110 acquires a target image, identifies one or more medicines included in the target image, and acquires, based on a result obtained by identifying the one or more medicines, stock information for each medicine type included in the target image. According to an example embodiment, the electronic apparatus 110 may identify the one or more medicines included in the target image by identifying one or more feature components included in the target image and acquiring type information of a medicine related to a first feature component included in the identified one or more feature components.

According to an example embodiment, the electronic apparatus 110 may transmit the acquired stock information to the manager terminal 120.

A manager may correspond to a subject who manages medicine stock. The manager terminal 120 may receive an input from the manager and the like or receive information from the electronic apparatus 110 and the like and perform an operation corresponding thereto. According to an example, the manager terminal 120 may acquire the target image and transmit the target image to the electronic apparatus 110. According to another example embodiment, the manager terminal 120 may receive the stock information for each medicine type from the electronic apparatus 110 and provide the stock information to the manager through an output device such as a screen.

Meanwhile, the manager may include a seller who sells medicines to customers or a provider (for example, a vendor such as a production company) who provides medicines to sellers of the medicines, but is not limited thereto.

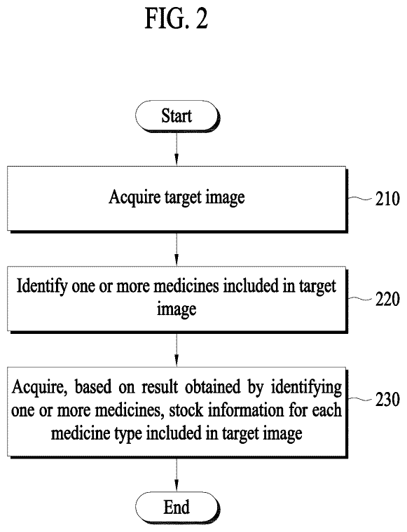

Descriptions of the electronic apparatus 110 and the manager terminal 120 will be described in more detail below in FIGS. 2 to 9.

Operations related to a series of methods of providing information according to various example embodiments may be implemented by a single physical device or implemented in a way in which a plurality of physical devices are coupled organizationally. For example, a portion of components included in the system that manages stock information may be implemented by one physical device, and a remaining portion may be implemented by another physical device. For example, the one physical device may be implemented as a portion of the electronic apparatus 110, and the another physical device may be implemented as a portion of the manager terminal 120 or a portion of an external device.

In some cases, each of components included in the system that manages stock information may be disposed to be distributed in different physical devices, and the distributed components may be coupled organizationally to be implemented to perform functions and operations of the system that manages stock information. For example, the electronic apparatus 110 of the present specification may include at least one sub-device. Partial operations described to be performed by the electronic apparatus 110 may be performed by a first sub-unit, and other partial operations may be performed by a second sub-unit.

FIG. 2 is a diagram for describing a method of acquiring, by an electronic apparatus, stock information of medicines according to an example embodiment.

Referring to FIG. 2, in operation 210, the electronic apparatus 110 according to an example embodiment acquires a target image. According to an example embodiment, the electronic apparatus 110 may acquire the target image by using its own photographing function. According to an example embodiment, the electronic apparatus 110 may acquire the target image by using directly connected photographing equipment. According to an example embodiment, the electronic apparatus 110 may receive the target image from another device (for example, the manager terminal 120). In addition, there may be various example embodiments related to an operation of acquiring the target image.

In operation 220, the electronic apparatus 110 may identify one or more medicines included in the target image. According to an example embodiment, the electronic apparatus 110 may identify one or more feature components included in the target image and identify one or more medicines included in the target image by acquiring type information of a medicine related to a first feature component included in the one or more feature components. In relation to this, the one or more feature components may include an object that may be a target to be identified, which may include, for example, logo information of a medicine.

Meanwhile, according to an example embodiment, the electronic apparatus 110 may not necessarily perform “an operation of acquiring type information of a medicine related to a first feature component” for all of the one or more feature components. For example, when the electronic apparatus 110 identifies five feature components that seem to be related to a medicine in the target image, and when the related medicine is specified based on two feature components among them, the operation of acquiring type information of the related medicine may not be performed for the remaining three feature components.

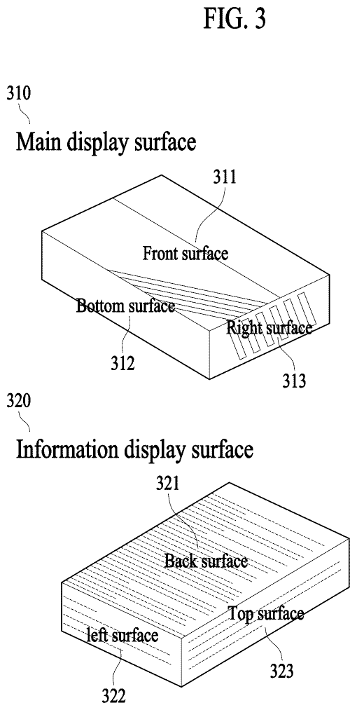

FIGS. 3 to 5 may be further referred to for understanding the one or more feature components in more detail. An example embodiment of an outer package of a medicine is illustrated in FIG. 3. Referring further to FIG. 3, the outer package of the medicine according to an example embodiment may be divided into a main display surface 310 and an information display surface 320. For example, the main display surface 310 may include a front surface 311, a bottom surface 312, and a right surface 313, and the information display surface 320 may include a back surface 321, a left surface 322, and a top surface 323. Meanwhile, whether which surface is included in the main display surface 310 and the information display surface 320 is merely an example. An example of FIG. 3 does not limit the scope of the present disclosure. Furthermore, covering a medicine in such a cube-shaped package is also merely an example for understanding, and it should be understood that, in addition to such a form of packaging, medicines may be packaged in various forms and methods in the present disclosure, such as packaging by a cylindrical container, cellophane packaging, or press-through-pack (PTP) packaging.

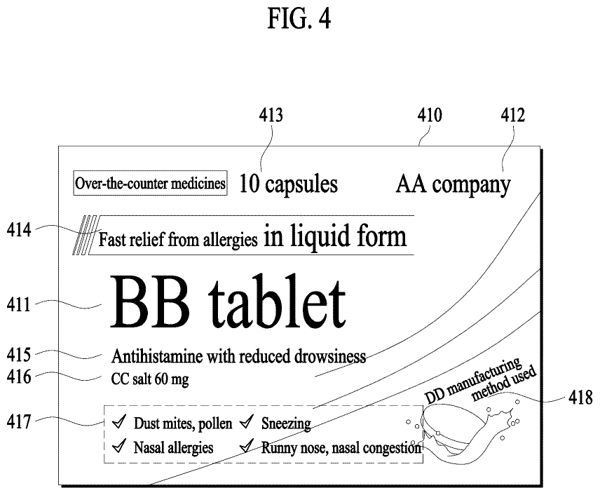

An example of the main display surface 310 is illustrated in more detail in reference numeral 410 of FIG. 4. Referring further to FIG. 4, a main display surface according to an example may include various pieces of information such as medicine name information (“BB tablet” 411), medicine manufacturer information (“AA company” 412), medicine classification and quantity information (“over-the-counter medicines” and “10 capsule” 413), medicine efficacy information (“fast relief from allergies in liquid form” 414 and “antihistamine with reduced drowsiness” 415), medicine ingredients and strength information (“CC salt 60 mg” 416), symptom information that may be a target of the medicine (“dust mites, pollen,” “sneezing,” “nasal allergies,” “runny nose, nasal congestion” 417), and information on a manufacturing method used for the medicine (“DD manufacturing method used” 418).

As such, at least a portion of various pieces of information included in the main display surface may be included in “one or more feature components”. For example, the electronic apparatus 110 may identify information such as “BB tablet,” “AA company,” and “CC salt 60 mg” included in the target image and, based on this, acquire type information of a corresponding medicine.

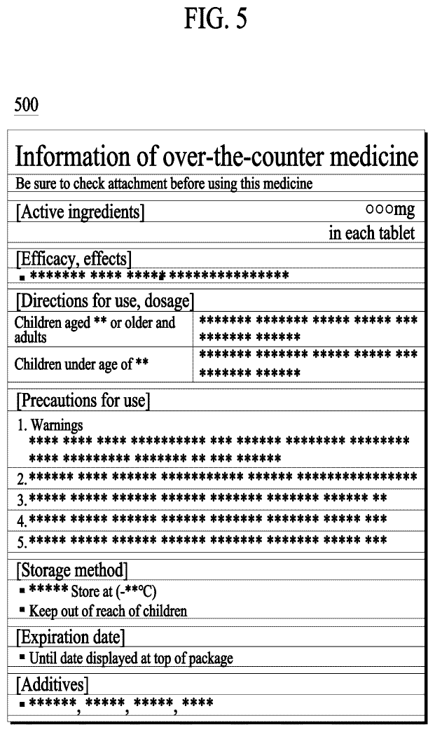

An example of the information display surface 320 is illustrated in more detail in reference numeral 500 of FIG. 5. Referring further to FIG. 5, the information display surface according to an example may include various pieces of information, such as active ingredients, efficacy, effects, directions for use, dosage, precautions for use, a storage method, an expiration date, and additives.

According to an example embodiment, at least a portion of various pieces of information included in the information display surface may be further included in the “one or more feature components”. For example, the electronic apparatus 110 may identify information such as “active ingredients,” “efficacy and effects,” and “precautions for use” and, further based on this, acquire type information of a corresponding medicine.

In particular, when there is an image having a main display surface that is not revealed or a main display surface whose information is difficult to be identified among images of medicines included in a target image, it is possible to actively identify and use information included in an information display surface for acquiring type information of a medicine related to the image.

Referring back to FIG. 2, the electronic apparatus 110 according to an example embodiment may acquire type information of a medicine related to a first feature component by comparing the first feature component with one or more comparative images. For example, by comparing the first feature component including text of “AA company” with a comparative image including text of “BB company,” a comparative image including text of “CC company,” and a comparative image including text of “AA company,” the electronic apparatus 110 may relate type information of a medicine related to the first feature component to type information of a medicine related to the comparative image including the text of “AA company”. In relation to this, even when it is difficult to specify a type of a medicine based on a single feature component (for example, when there are various types of medicines produced in “AA company”), it is possible to gradually narrow down candidates of medicine types that may be related to the corresponding feature component. Accordingly, it may be understood that the electronic apparatus 110 repetitively performs such a comparison operation for a plurality of feature components to specify a type of a related medicine.

The comparative images may include one or more images stored in advance, which are not necessarily stored in the electronic apparatus 110 (for example, the images may be stored in an external database), but may be stored in a state that may be brought by the electronic apparatus 110 by necessity.

According to an example embodiment, the electronic apparatus 110 may acquire types of medicines in advance that are handled or likely to be handled in a place in which the target image is photographed (that is, a place as a target of stock management), further determine whether an image belongs to the types, and select a comparative image to be compared with the first feature component. In other words, when there is a medicine that is not handled and not likely to be handled in a corresponding place (for example, a pharmacy), by preventing the medicine from being a target to be compared with feature components included in the target image, it is possible to increase accuracy of stock checking and save costs (for example, resource consumption) for stock checking.

According to an example embodiment, the electronic apparatus 110 may determine one or more attributes related to the first feature component and, based on the determined one or more attributes, select one or more comparative images among a plurality of images included in a medicine image group. In a related example, the electronic apparatus 110 may determine a type of a feature component corresponding to the first feature component and perform a comparison with a comparative image belonging to a corresponding type. For example, the electronic apparatus 110 may determine that the first feature component is a type of efficacy information such as “fast relief from allergies in liquid form” and “antihistamine with reduced drowsiness” and perform a comparison with a comparative image having an identical type of efficacy information.

According to an example, the electronic apparatus 110 may identify logo information included in the first feature component and, based on the identified logo information, determine one or more attributes related to the first feature component by performing a comparison with a comparative image belonging to a logo. The logo information may be understood as information including various types of logos such as a logo of an importer, a logo of a distributor, and a logo of a manufacturer. The logo information may be appropriate for identifying a medicine by being compared with a comparative image since the logo information may include, in addition to content of text, various pieces of attribute information such as a text color, a text font, a background color except for text, which will be described below, and may further include attribute information such as a shape or position of a logo.

According to an example embodiment, the electronic apparatus 110 may determine a more specific attribute beyond a type of a feature component and may perform a comparison with a comparative image having the corresponding attribute. For example, the electronic apparatus 110 may identify that the first feature component has a text color of white, a text font of Gulim, and a background color of purple except for text and may perform a comparison with a comparative image having a matching text color, text font, and background color.

Meanwhile, according to a photographing environment, or according to various types of image processing (for example, image format conversion for transmission between devices), a color included in an image may change. As such, as a method for considering that an image color may be changed, there may be various example embodiments such as excluding a color from attributes for filtering comparative images, selecting an image in consideration of a color as a comparative image having a corresponding attribute when color matching is not accurately exact but falling within a certain similarity range, or selecting a comparative image by inferring a process in which a color is changed by a photographing environment or image processing (for example, after inferring the process in which the color is changed, acquiring an original color by applying the process in reverse).

Meanwhile, for convenience of understanding, “color” is described as an example, but this does not necessarily mean for other attributes that a comparative image having a “matching” attribute is required to be selected. It should be understood that the color-related example may be used as an example of an operation of selecting an appropriate comparative image.

As such, instead of performing a comparison between the target image and the plurality of images included in the medicine image group one by one, by performing, based on one or more attributes related to the first feature component, an operation of selecting a comparative image to be a target to be compared, it is possible to significantly reduce a repetition number of the operation of comparing the target image with other images, thereby having a technical effect of greatly reducing costs (resources and time) for identifying one or more medicines included in the target image.

According to an example embodiment, the electronic apparatus 110 may perform optical character recognition (OCR) for one or more of the target image and comparative images and, based on a result thereof, compare two images. For example, even when the target image and a comparative image do not seem to have similar colors, fonts, and materials, if the target image and the comparative image have something in common in that they have text of “BB tablet,” “AA company,” and “DD manufacturing method,” the target image and the comparative image may be determined as an identical type of medicines.

According to an example embodiment, the electronic apparatus 110 may input the first feature component and one or more comparative images to a pre-trained machine learning model and compare, by acquiring an output of the machine learning model, the first feature component and the one or more comparative images. It may be understood that the machine learning model is pre-trained based on a plurality of medicine images. Furthermore, according to the method of the present disclosure, based on a result obtained by identifying one or more medicines included in the target image and a result obtained by acquiring stock information for each medicine type included in the target image, the electronic apparatus 110 may further acquire one or more medicine images (which may be the target image itself or an image acquired based on the target image) and further train the machine learning model further based on the acquired one or more medicine images.

According to an example embodiment, the electronic apparatus 110 may appropriately process or modify the first feature component for increasing an identification possibility of medicines. According to a related example, the electronic apparatus 110 may acquire gradient information related to the first feature component and rotate the first feature component based on the acquired gradient information. The gradient information may include a two-dimensional gradient and may include a three-dimensional gradient.

According to another example, the electronic apparatus 110 may acquire distortion information related to the first feature component and correct the first feature component based on the acquired distortion information. Distortion related to the first feature component may include, for example, elements such as shape distortion, excessive gathering of light, and shadow formation, which may occur due to limitations of an essential structure of a camera. As an example of an operation of correcting the first feature component, the electronic apparatus 110 may perform warping for the first feature component.

The above-mentioned “operation of appropriately processing or modifying the first feature component” may be understood as an operation of determining whether there is a gradient or distortion for each feature component by an image itself (that is, based on a shape or color of the image itself, it is possible to determine whether there is a gradient, or a hidden portion by a shadow).

Meanwhile, the electronic apparatus 110 according to an example embodiment may acquire photographing environment information of the target image and transform the target image based on the photographing environment information. As such, by transforming the target image, it is possible to increase the identification possibility of one or more medicines and accuracy of the identification. The operation of transforming the target image based on the photographing environment information (unlike the operation of appropriately processing or modifying the first feature component) may be understood as an operation of transforming the target image based on external environment factors rather than the shape or color of the image itself.

The photographing environment information may include at least one or more pieces of product information of a camera for photographing the target image, setting information of the camera when photographing the target image, distance information between the camera and a target to be photographed when photographing the target image, and angle information between the camera and the target to be photographed when photographing the target image. Based on one or more pieces of the product information of the camera and the setting information of the camera, the electronic apparatus 110 may determine a type of distortion and a degree of the distortion that may occur by the camera and appropriately transform the target image such that the distortion is corrected. Likewise, based on one or more pieces of the distance information between the camera and the target to be photographed when photographing the target image and the angle information between the camera and the target to be photographed when photographing the target image, the electronic apparatus 110 may determine a tilt degree of a medicine image included in the target image and a size of the image and, based on this, appropriately transform the target image.

Meanwhile, the above-mentioned “operation of transforming the target image” may include “an operation of transforming partial components included in the target image,” and a method of the transformation may be different for each component. For example, when the camera photographs a plurality of medicines, since there may be a difference in the tilt degree or a distance from the camera for each medicine, transformation according to different methods for each medicine may be performed.

The electronic apparatus 110 may compare the transformed target image (or a component included in the target image) with the above-mentioned comparative images.

According to an example embodiment, the electronic apparatus 110 may acquire photographing environment information of the target image and transform the comparative images based on the photographing environment information. It may be expected that the comparative images are likely to have a greater image quality compared to the target image or have a degree of tilt or distortion less than that of the target image, and to be compared with the comparative images, leaving the target image as it is may reduce complexity of procedures. Therefore, by transforming the comparative images, it is possible to increase the identification possibility of one or more medicines and the accuracy of the identification. Meanwhile, when transforming the comparative images, it may be understood that the transformation of the comparative images may be performed by applying the above-mentioned “operation of transforming the target image” in reverse (for example, when it was supposed to rotate the target image by 20 degrees clockwise, instead, rotating the comparative images by 20 degrees counterclockwise), but the method of the present disclosure is not necessarily limited thereto.

The electronic apparatus 110 may compare the target image (or a component included in the target image) with the transformed comparative images.

By transforming the target image or the comparative images, a technical effect of minimizing effects of various hindrances (for example, tilt, distortion, and magnification or reduction of size) that may occur when identifying medicines included in the target image may be obtained.

There may be various example embodiments in relation to a specific method of acquiring the photographing environment information of the target image by the electronic apparatus 110. According to an example embodiment, the electronic apparatus 110 may receive the photographing environment information. According to an example embodiment, the electronic apparatus 110 may receive information that is a basis of the photographing environment information. For example, the electronic apparatus 110 may receive position information of the camera and, based on this, calculate a distance and an angle from targets to be photographed so as to acquire the photographing environment information.

According to an example embodiment, the electronic apparatus 110 may generate the photographing environment information by using pre-acquired information in relation to a portion of the targets to be photographed. For example, for partial medicines among the targets to be photographed, when the electronic apparatus 110 accurately recognizes sizes and shapes of the partial medicines, by comparing the accurately recognized sizes and shapes with a size and shape of a medicine photographed by the camera, it is possible to determine a distance from the camera to the medicine and an angle between the camera and the medicine. In other words, it may be understood that the electronic apparatus 110 acquires photographing environment information based on the targets to be photographed whose relevant information is already accurately recognized, and by using the photographing environment information thereafter, transforms the target image (or at least a portion of the targets to be photographed included in the target image) again. (In other words, it is possible to transform the target image to be photographed based on the photographing environment information and acquire the photographing environment information based on the target image to be photographed. That is, information acquisition may be performed in both ways.)

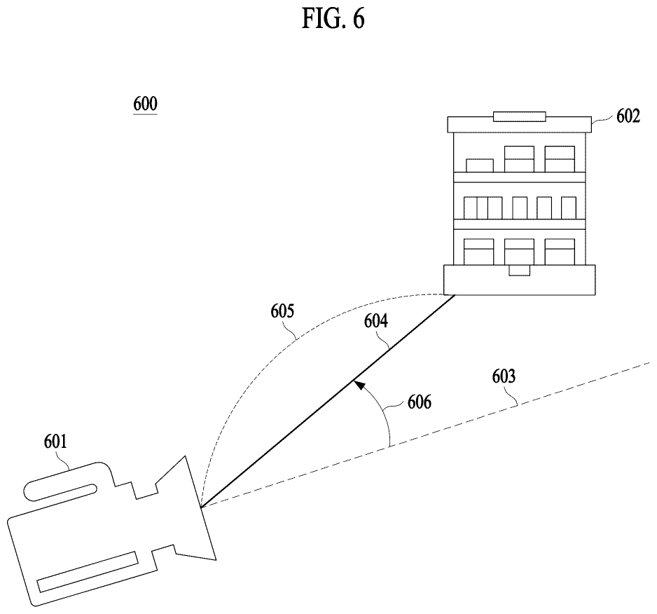

An example of the photographing environment information of the target image is illustrated in FIG. 6. Referring further to an example 600 of FIG. 6, the photographing environment information may include one or more pieces of product information of a camera 601 and setting information of the camera 601.

In addition, the photographing environment information according to an example embodiment may include distance information 605 between the camera 601 and a target to be photographed 602. In relation to this, the distance information 605 according to an example embodiment may include a length of a line 604 connecting the camera 601 and the target to be photographed 602, but the scope of the present disclosure is not necessarily limited thereto.

In addition, the photographing environment information according to an example embodiment may include angle information 606 between the camera 601 and the target to be photographed 602. In relation to this, the angle information 606 according to an example embodiment may include an angle formed between a reference line 603 in a forward-facing direction of the camera 601 and the line 604 connecting the camera 601 and the target to be photographed 602, but the scope of the present disclosure is not necessarily limited thereto.

Referring back to FIG. 2, in operation 230, the electronic apparatus 110 may acquire stock information for each medicine type included in a target image based on a result obtained by identifying one or more medicines. A medicine whose type is determined by the electronic apparatus 110 may be included in medicines directly shown in the target image, but is not limited thereto. For example, the electronic apparatus 110 according to an example embodiment may determine types of a plurality of medicines existing in a space corresponding to the target image. It may be understood that the plurality of medicines “existing in the space” corresponding to the target image include medicines having at least a portion directly shown in the target image and also include medicines that are assumed to be hidden although at least a portion of the medicines is not directly shown.

Meanwhile, in relation to a specific method of assuming that a medicine exists in a hidden space, there may be an example of estimating how much more medicines are able to be stored in an inner space based on length information such as a depth or inside volume information of a place in which the medicine is stored (shelves or medicine cabinets). However, the scope of the present disclosure is not limited to such an example.

The electronic apparatus 110 may acquire position information of a medicine whose type has been determined. According to an example embodiment, the electronic apparatus 110 may acquire the position information of the medicine based on one or more of a relative position relationship with a place storing the medicine and a relative position relationship with other medicines. For example, when a shelf storing medicines has a physical structure capable of being divided into several sections, the electronic apparatus 110 may designate each section of the shelf as a first section, a second section, ..., and an n^{th} section and may acquire position information of medicines as “inside the first section,” “inside the second section,” ..., and “inside the n^{th} section.” As another example, the electronic apparatus 110 may determine relative positions between medicines, set an x-coordinate of a medicine at a leftmost position to “1” to set each x-coordinate increasing one by one in order of positions toward a right side based on the leftmost medicine, and likewise, set a y-coordinate of a medicine at a bottommost position to “1” to set each y-coordinate increasing one by one in order of positions toward an upper side based on the bottommost medicine. In addition, there may be various examples of setting position information of medicines, and a specific example does not limit the scope of the present disclosure.

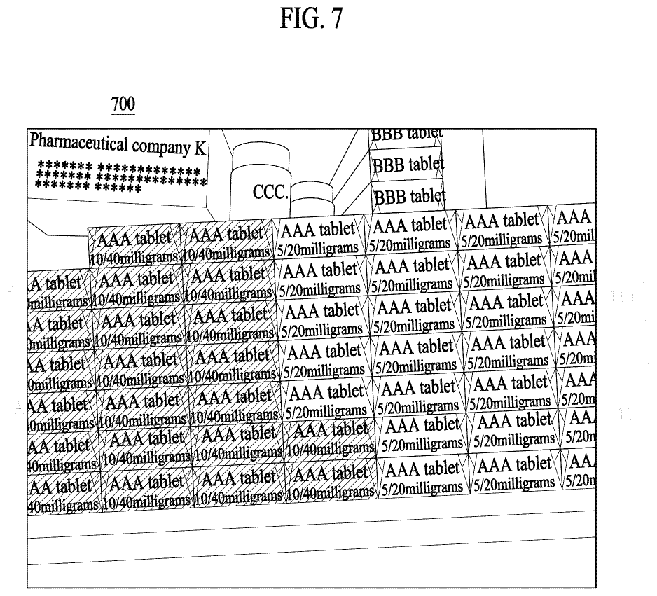

In FIG. 7, examples of a method in which medicines are stored are illustrated. Referring further to an example 700 of FIG. 7, outer packaging of each medicine may include information of each name and strength. In an example of FIG. 7, there are “AAA tablet 10/40 milligrams ” and “AAA tablet 5/20 milligrams” individually. Similarly, even when medicines have an identical name, they may have different quantities or strengths depending on their packaging units, which may be identified through the outer packaging. In addition, in an example of FIG. 7, it may be identified that packaging of the “AAA tablet 10/40 milligrams” and packaging of the “AAA tablet 5/20 milligrams” have different colors. Accordingly, it may be identified that each packaging unit has a different quantity and strength based on an attribute such as a color or shape of the outer packaging.

Meanwhile, for convenience of understanding, the description of “different quantities or strengths” was used, but there may be various example embodiments. For example, medicines having an identical name may have different efficacy, may be obtained by different manufacturing methods, or may be used for different symptoms. For such example embodiments, the above description may be similarly applied.

In addition, tablet AAA and tablet BBB are separated in individual cube-shaped packaging, but there may be several example embodiments related to a specific packaging method. For example, like “pharmaceutical company K” at a left upper end, several medicines by an identical manufacturer are stored in a large group together, and like “CCC” at a left center, medicines may be stored in a cylindrical container.

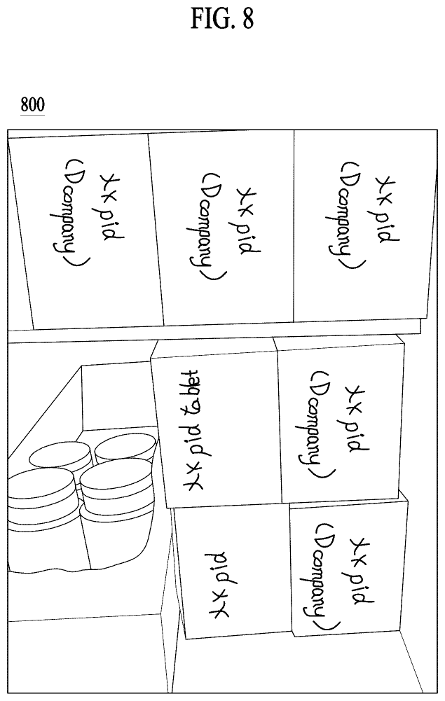

Meanwhile, according to an example embodiment in relation to a packaging method, information for specifying a medicine may be handwritten on outer packaging of the medicine. Referring further to an example 800 of FIG. 8, it may be identified that information such as “XXpid (D company),” “XXpid,” or “XXpid tablet” is handwritten. As such, when information is handwritten, the above-mentioned method of “performing OCR and, based on a result thereof, comparing a target image and comparative images” may be particularly useful. In relation to this, it may be understood that handwritten information may be more effective for determining a type of a medicine by determining and comparing substantial content since the information is not included in unified printed packaging.

Referring back to FIG. 2, the electronic apparatus 110 according to an example embodiment may acquire three-dimensional (3D) modeling information corresponding to a target image based on acquired stock information. As such, by acquiring the 3D modeling information, stock management may be performed thereafter by a method of correcting the stock information when it is detected that a medicine is brought in or taken out. Compared to a method of checking stock directly and continuously, the method of detecting a medicine brought in or taken out may be more desirable in terms of costs and accuracy. In addition, for direct checking of stock, a camera having relatively great performance (for example, capable of generating a high-resolution image) is required. However, a task of detecting a medicine brought in or taken out may be performed by a camera having relatively low performance. Accordingly, the method of detecting a medicine brought in or taken out may be understood to have greater accessibility (especially for small businessmen).

According to a more specific example embodiment, the electronic apparatus 110 may generate one or more geometric models corresponding to at least a portion of a plurality of medicines existing in a space corresponding to the target image and may perform a texture mapping process for the generated one or more geometric models to acquire 3D modeling information. As an example of an operation of generating a geometric model, the electronic apparatus 110 may model outer packaging of a medicine as a cube-shaped mesh having a size on an x-axis, a y-axis, and a z-axis or model outer packaging of a medicine as a cylinder-shaped mesh having a radius of “r” and a height of “l”. Medicine types (for example, codes) may be mapped in the geometric model.

The texture mapping process may include a process of applying texture, such as an image and a pattern, to a 3D model to provide various attributes such as colors, texture, and details. As a specific method of the texture mapping process, the electronic apparatus 110 may perform the texture mapping process by performing an unwrapping process of unfolding each portion of the 3D model to be arranged in a two-dimensional (2D) space, a process of producing texture (to represent, for example, colors, reflection of light, transparency, and contrast) by generating an image using graphics software or by using a pre-generated image, and a mapping process of mapping texture to an unwrapped map to apply the texture to each portion of the 3D model. However, this is only a specific example for helping understanding of the texture mapping process, and the scope of the present disclosure is not limited to such an example.

In the present disclosure, as an example of acquiring position information of medicines, an example of recognizing an x-coordinate and a y-coordinate is described, and for 3D modeling, an example of a cube having a size on the x-axis, the y-axis, and the z-axis is described. However, in such examples, for convenience of description, a coordinate or axis is directly mentioned, but actually, it should be understood that examples of defining or expressing such information as a matrix or vector also fall within the scope of the present disclosure.

According to an example embodiment, the electronic apparatus 110 may acquire storage environment information of one or more medicines, and the electronic apparatus 110 may acquire stock information based on the acquired storage environment information. The storage environment information may include one or more pieces of attribute information related to a storage part for storing the one or more medicines. The one or more pieces of attribute information according to an example may include one or more pieces of information for product identification of the storage part, shape information of the storage part, size information of the storage part, and volume information of the storage part. The information for product identification of the storage part may include, for example, information such as a manufacturer and a product name of the storage part (that is, information such as a manufacturer of a shelf and a name of the shelf).

Based on one or more pieces of the shape information, the size information, and the volume information of the storage part, the electronic apparatus 110 may determine a size and shape of a 3D space in which the storage part is able to store articles such as medicines. The determined size and shape of the 3D space may be used for acquiring the above-mentioned 3D modeling information. Or, based on the determined size and shape of the three-dimensional space, the electronic apparatus 110 may estimate a number of medicines that can be stored more in an inner space of a place in which medicines are stored (related to the above-mentioned operation of determining types of a plurality of medicines “existing in the space” corresponding to a target image).

Meanwhile, in addition to the above-mentioned shape information, size information, and volume information of the storage part, various pieces of attribute information, such as attribute information related to whether the storage part maintains its shape well, attribute information related to whether the storage part is fixable to a wall surface (or whether the storage part is movable inside a wall surface), attribute information related to whether the storage part is fixable to a fake wall, attribute information related to whether the storage part has a fixed position relationship with other shelves, and attribute information related to whether the storage part is easily disassembled.

According to an example embodiment, the electronic apparatus 110 may acquire suggestion information related to a storage method of one or more medicines based on storage environment information and stock information. For example, the electronic apparatus 110 may suggest a change in a storage position and a piling method such that a medicine that is actively brought in or taken out is stored at a position easily accessible by a person.

According to an example embodiment, the electronic apparatus 110 may compare the stock information acquired through operation 230 with pre-existing computerized stock information. When there is a discrepancy between the stock information acquired through operation 230 and the pre-existing computerized stock information, the electronic apparatus 110 may provide information for notifying the discrepancy. The information for notifying the discrepancy may be provided to the manager terminal 120, but a target to be provided is not limited to the manager terminal 120. A specific example of a case when there is a discrepancy includes a case in which an item that does not exist in computerized stock is identified in a target image and a case in which a quantity of an item A in computerized stock is different from a quantity of the item A in the target image, but a specific example does not limit the scope of the present disclosure.

As such, by comparing the stock information with the pre-existing computerized stock information, whether the stock information acquired by the electronic apparatus 110 and the pre-existing computerized stock information are accurate may be verified individually.

Meanwhile, according to an example embodiment (especially in an environment in which it is difficult to use a camera with great performance), the electronic apparatus 110 may easily infer a type of a medicine included in the target image with reference to the computerized stock and determine a change in stock more easily. As described above in relation to the “operation of acquiring 3D modeling information,” whereas a camera with relatively great performance (for example, capable of generating a high-resolution image) is required for direct checking of stock, a task of just detecting a medicine brought in or taken out may be performed by a camera with relatively low performance. Accordingly, it may be understood that a method of determining a change in stock more easily with reference to computerized stock has great accessibility.

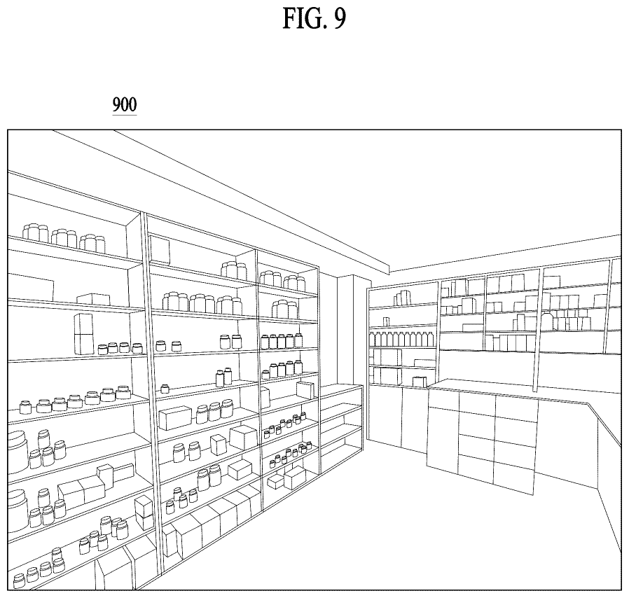

FIG. 9 is a diagram for describing 3D modeling information according to an example embodiment.

Referring to an example 900 of FIGS. 9, 3D modeling information according to an example embodiment may include a plurality of geometric models reflecting each shape of outer packaging, and a medicine type may be mapped for at least a portion of the plurality of geometric models. Furthermore, texture may be mapped for at least a portion of the plurality of geometric models.

The 3D modeling information according to an example embodiment may reflect a position in which each geometric model is stored on a storage part. Furthermore, the 3D modeling information according to an example embodiment may reflect one or more pieces of attribute information related to the storage part. According to an example embodiment, the 3D modeling information may reflect information on an empty space in which a medicine is not stored yet in the storage part and thus facilitate a task of further storing medicines.

The 3D modeling information according to an example 900 of FIG. 9 may be expressed as an image reflecting various pieces of information, but the 3D modeling information is not necessarily expressed as an image. For example, the 3D modeling information may include one or more pieces of information expressed in text, such as {medicine 1: coordinates (1, 0, 3) and texture (red, no reflection, no transparency, and high contrast)}. In addition, expression forms of the 3D modeling information may be diverse, and a specific form does not limit the scope of the present disclosure.

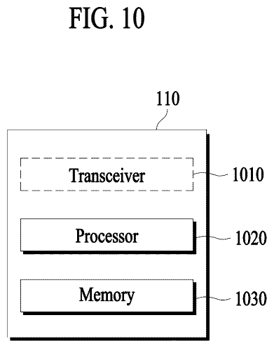

FIG. 10 is an exemplary view of a configuration of an electronic apparatus according to an example embodiment.

Referring to FIG. 10, the electronic apparatus 110 may include a processor 1020 and a memory 1030, and may further include a transceiver 1010 according to an example embodiment. The electronic apparatus 110 may be connected to the manager terminal 120 and other external devices through the transceiver 1010, and may exchange data.

The processor 1020 may include at least one device described above through FIGS. 1 to 9, or may perform at least one method described above through FIGS. 1 to 9. The memory 1030 may store information for performing at least one method described above through FIGS. 1 to 9. The memory 1030 may be a volatile memory or a non-volatile memory.

The processor 1020 may execute a program and control the electronic apparatus 110 for providing information. A code of the program executed by the processor 1020 may be stored in the memory 1030.

In addition, the electronic apparatus 110 of an example embodiment may further include an interface capable of providing information to users.

According to the present disclosure, an apparatus of processing information and a method thereof may reduce costs and effort for managing medicine stock.

The effects of the invention are not limited to those described above, and other effects not explicitly described may be clearly understood by those skilled in the art from the scope of the claims.

Meanwhile, disclosed are example embodiments of the present disclosure in the present specification and drawings, it should be understood that specific terms used herein are merely for the purpose of easily describing the technical content of the present disclosure and facilitating understanding of the present disclosure, and are not intended to limit the scope of the present disclosure. It will be apparent to those skilled in the art that, in addition to the embodiments disclosed herein, various modifications based on the technical spirit of the present disclosure may also be implemented.

A recommendation server, an electronic apparatus, or a terminal according to the above-mentioned example embodiments may include a processor, a memory that stores and executes program data, a permanent storage such as a disk drive, a communication port that communicates with an external device, and a user object device such as a touch panel, a key, and a button. Methods implemented as software modules or algorithms may be stored in a computer-readable recording medium as computer-readable codes or program instructions executable by the processor. Here, the computer-readable recording medium may include a magnetic storage medium (for example, a read-only memory (ROM), a random-access memory (RAM), a floppy disk, and a hard disk) and an optical reading medium (for example, a CD-ROM and a digital versatile disc (DVD)). The computer-readable recording medium may be distributed across computer systems connected through a network, and computer-readable codes may be stored and executed in a distributed manner. The medium may be readable by a computer, stored in the memory, and executed by the processor.

The present example embodiments may be represented by functional blocks and various processing steps. These functional blocks may be implemented by various numbers of hardware and/or software configurations that execute specific functions. For example, the present example embodiments may adopt integrated circuit configurations such as a memory, a processor, a logic circuit, and a look-up table that may execute various functions by control of one or more microprocessors or other control devices. Similarly to that components may be executed by software programming or software components, the present example embodiments may be implemented by programming or scripting languages such as C, C++, Java, and assembler language, and Python including various algorithms implemented by combinations of data structures, processes, routines, or of other programming configurations. Functional aspects may be implemented by algorithms executed by one or more processors. In addition, the present example embodiments may adopt the related art for electronic environment setting, signal processing, and/or data processing. The terms “mechanism,” “component,” “means,” and “configuration” may be widely used and are not limited to mechanical and physical elements. These terms may include meaning of a series of routines of software in association with the processor.

The above-described example embodiments are merely examples, and other example embodiments may be implemented within the scope of the following claims.

What is claimed is:

1. A method of acquiring information from an electronic apparatus, the method comprising:
acquiring a target image;
identifying one or more medicines included in the target image; and
acquiring, based on a result obtained by identifying the one or more medicines, stock information for each medicine type included in the target image.

2. The method of claim 1, wherein the identifying of the one or more medicines comprises:
identifying one or more feature components included in the target image; and
acquiring type information of a medicine related to a first feature component included in the one or more feature components.

3. The method of claim 2, wherein the acquiring of the type information of the medicine related to the first feature component comprises comparing the first feature component with one or more comparative images.

4. The method of claim 3, wherein the comparing of the first feature component with the one or more comparative images comprises:
inputting the first feature component and the one or more comparative images to a pre-trained machine learning model; and
acquiring an output of the machine learning model, and
wherein the machine learning model is pre-trained based on a plurality of medicine images.

5. The method of claim 3, wherein the comparing of the first feature component with the one or more comparative images comprises:
determining one or more attributes related to the first feature component; and
selecting, based on the one or more attributes, the one or more comparative images from a plurality of images included in a medicine image group.

6. The method of claim 5, wherein the determining of the one or more attributes comprises identifying logo information included in the first feature component.

7. The method of claim 2, wherein the acquiring of the type information of the medicine related to the first feature component comprises:
acquiring gradient information related to the first feature component; and
rotating the first feature component based on the gradient information.

8. The method of claim 2, wherein the acquiring of the type information of the medicine related to the first feature component comprises:
acquiring distortion information related to the first feature component; and
correcting the first feature component based on the distortion information, and
wherein the correcting of the first feature component comprises performing warping for the first feature component.

9. The method of claim 1, further comprising acquiring photographing environment information of the target image,
wherein the identifying of the one or more medicines comprises transforming the target image based on the photographing environment information.

10. The method of claim 9, wherein the photographing environment information includes one or more pieces of:
product information of a camera for photographing the target image,
setting information of the camera when the target image is photographed,
distance information between the camera and a target to be photographed when the target image is photographed, and
angle information between the camera and the target to be photographed when the target image is photographed.

11. The method of claim 1, wherein the acquiring of the stock information comprises:
determining types of the plurality of medicines existing in a space corresponding to the target image; and
acquiring position information of the plurality of medicines.

12. The method of claim 1, further comprising acquiring, based on the stock information, three-dimensional (3D) modeling information corresponding to the target image.

13. The method of claim 12, wherein the acquiring of the 3D modeling information comprises:
generating one or more geometric models corresponding to at least a portion of the plurality of medicines existing in a space corresponding to the target image; and
performing a texture mapping process for the one or more geometric models.

14. The method of claim 1, further comprising acquiring storage environment information of the one or more medicines, and
wherein the electronic apparatus acquires the stock information based on the storage environment information.

15. The method of claim 14, wherein the storage environment information includes one or more pieces of attribute information related to a storage part for storing the one or more medicines, and
wherein the one or more pieces of attribute information include one or more pieces of:
information for product identification of the storage part,
shape information of the storage part,
size information of the storage part, and
volume information of the storage part.

16. The method of claim 14, further comprising acquiring, based on the storage environment information and the stock information, suggestion information related to a storage method of the one or more medicines.

17. The method of claim 1, further comprising comparing the stock information with pre-existing computerized stock information.

18. The method of claim 17, further comprising notifying a discrepancy when there is the discrepancy between the stock information and the pre-existing computerized stock information.

19. A non-transitory computer-readable recording medium recording a program for executing the method of claim 1 in a computer.

20. An electronic apparatus acquiring information, the electronic apparatus comprising:
a memory configured to store an instruction and a processor,
wherein the processor, which is connected to the memory, is configured to:
acquire a target image;
identify one or more medicines included in the target image; and
acquire, based on a result obtained by identifying the one or more medicines, stock information for each medicine type included in the target image.
